(19) United States
(12) Patent Application Publication
(10) Pub. No.: US 2024/0269413 A1
(43) Pub. Date: Aug. 15, 2024

(54) MASK ASSEMBLY FOR USE WITH A CONTINUOUS POSITIVE AIRWAY PRESSURE MACHINE

(71) Applicant: BRANDON SCOTT MCDONALD, Fremont, MI (US)

(72) Inventor: BRANDON SCOTT MCDONALD, Fremont, MI (US)

(21) Appl. No.: 18/441,128

(22) Filed: Feb. 14, 2024

Related U.S. Application Data
(60) Provisional application No. 63/445,634, filed on Feb. 14, 2023.

(51) Int. Cl. A61M 16/06

(52) U.S. Cl. CPC A61M 16/0616; A61M 2205/0216; A61M 2205/3331; A61M 2210/0625

(57) ABSTRACT

A mask assembly is provided with a substrate sized to cover a mouth of a user. A plurality of ports is formed through the substrate. A valve seal is mounted to the substrate. The valve seal has a plurality of valves formed through the valve seal. A peripheral seal is mounted to the substrate to seal around the valve to seal the substrate and the valve seal to a face of the user around the mouth of the user.

### CROSS-REFERENCE TO RELATED APPLICATIONS

This application claims the benefit of U.S. provisional application Ser. No. 63/445,634 filed Feb. 14, 2023, the disclosure of which is hereby incorporated in its entirety by reference herein.

### TECHNICAL FIELD

Various embodiments relate to a mask assembly that is used in conjunction with continuous positive airway pressure (CPAP) machine.

### BACKGROUND

Continuous positive airway pressure (CPAP) therapy was introduced in the 1980s. The purpose of CPAP therapy is to provide continuous positive airway pressure to keep an airway open to avoid disruptions in a patient breathing while sleeping. Such disruptions are known as sleep apnea. CPAP therapy often includes CPAP machines, which are machines that generate a source of pressurized fluid, such as air, that is connected to a hose and the hose is connected to a mask. Masks vary and include full-face masks and nasal masks.

### SUMMARY

According to at least one embodiment, a mask assembly is provided with a substrate sized to cover a mouth of a user. A plurality of ports is formed through the substrate. A valve seal is mounted to the substrate. The valve seal has a plurality of valves formed through the valve seal. A peripheral seal is mounted to the substrate to seal around the valve to seal the substrate and the valve seal to a face of the user around the mouth of the user.

According to a further embodiment, the ports in the substrate are formed as plurality of apertures.

According to another further embodiment, the valves are formed through the valve seal as a plurality of apertures.

According to an even further embodiment, the plurality of apertures in the valve seal are not aligned with the plurality of ports in the substrate.

According to another even further embodiment, the plurality of apertures is formed through the valve seal as a plurality of slits. Each slit of the plurality of slits intersects another slit of the plurality of slits.

According to an even further embodiment, the intersecting slits provide a series of flaps. The valve seal is formed from a flexible material to permit elastic deformation of the flaps to open the valves, and retraction of the flaps to close the valves.

According to an even further embodiment, the valve seal is displaced along the substrate such that the substrate prevents deformation of the flaps in an egress direction of fluid flow, while permitting elastic deformation of the flaps in an ingress direction of fluid flow.

According to another further embodiment, a deformable member is attached to the substrate to permit shaping of the substrate to the face of the user.

According to another further embodiment, the substrate is sized to not cover a nose of the user.

According to another further embodiment, the valve seal is further provided with a diaphragm.

According to an even further embodiment, a retainer cooperates with the diaphragm and the substrate to retain the diaphragm to the substrate for removal and replacement of the diaphragm.

According to another embodiment, a mask assembly is provided with a substrate sized to be placed over a mouth of a user. A port is formed through the substrate. A valve seal is mounted to the substrate. The valve seal has a valve formed through the valve seal. The valve seal cooperates with the substrate to permit ingress of fluid through the port and the valve seal, while preventing egress of fluid through the valve seal and the port. A peripheral seal is mounted to the substrate to seal around the valve to seal the substrate and the valve seal to a face of the user around the mouth of the user.

According to a further embodiment, the port is further provided with a plurality of ports.

According to another further embodiment, the valve is further provided with a plurality of ports.

According to another further embodiment, the valve is formed through the valve seal as a plurality of intersecting slits. The intersecting slits provide a series of flaps. The valve seal is formed from a flexible material to permit elastic deformation of the flaps to open the valve, and retraction of the flaps to close the valve.

According to an even further embodiment, the valve seal is displaced along the substrate such that the substrate prevents deformation of the flaps in an egress direction of fluid flow, while permitting elastic deformation of the flaps in an ingress direction of fluid flow.

According to another further embodiment, the valve seal is further provided with a diaphragm.

According to an even further embodiment, a retainer cooperates with the diaphragm and the substrate to retain the diaphragm to the substrate for removal and replacement of the diaphragm.

According to another embodiment, a mask assembly is provided with a substrate sized to be placed over a mouth of a user. A port is formed through the substrate. A peripheral seal is mounted to the substrate to seal the substrate to a face of the user around the mouth of the user to provide an enclosed chamber around the mouth of the user. A check valve is mounted to the substrate to permit fluid ingress into the chamber, while preventing fluid egress from the chamber.

According to an even further embodiment, a retainer cooperates with the check valve and the substrate to retain the check valve to the substrate for removal and replacement of the check valve.

### BRIEF DESCRIPTION OF THE DRAWINGS

FIG. 1 is a front perspective view of a mask assembly according to an embodiment;

FIG. 2 is a rear perspective view of the mask assembly of FIG. 1;

FIG. 3 is front elevation view of a substrate of the mask assembly of FIG. 1;

FIG. 4 is a front elevation view of a diaphragm of the mask assembly of FIG. 1;

FIG. 5 is a front elevation view of the diaphragm and substrate of the mask assembly of FIG. 1;

FIG. 6 is a front elevation view of a seal of the mask assembly of FIG. 1;

FIG. 7 is a rear elevation view of the seal and substrate of the mask assembly of FIG. 1;

FIG. 8 is front elevation view of a bracket of the mask assembly of FIG. 1;

FIG. 9 is a front elevation view of the bracket and substrate of the mask assembly of FIG. 1;

FIG. 10 is a front elevation view of the bracket and substrate with a strap of the mask assembly of FIG. 1;

FIG. 11 is a side elevation view of a user wearing the mask assembly of FIG. 1;

FIG. 12 is a front elevation view of mask assembly of FIG. 1, illustrated partially assembled;

FIG. 13 is a side elevation view of a user wearing a mask assembly according to another embodiment, in conjunction with a CPAP nasal mask of a CPAP machine;

FIG. 14 is a front perspective view of the mask assembly of FIG. 13, according to another embodiment;

FIG. 15 is a front elevation view of a head rest of the mask assembly of FIG. 14;

FIG. 16 is another front perspective view of the mask assembly of FIG. 14;

FIG. 17 is a perspective view of a strap of the mask assembly of FIG. 16;

FIG. 18 is a top view of the mask assembly of FIG. 14;

FIG. 19 is a rear elevation view of the mask assembly of FIG. 14;

FIG. 20 is a front elevation view of a diaphragm of the mask assembly of FIG. 14;

FIG. 21 is a rear elevation view of the mask assembly of FIG. 14;

FIG. 22 is a front perspective view of the mask assembly of FIG. 14;

FIG. 23 is a top perspective view of the mask assembly of FIG. 14;

FIG. 24 is a front elevation view of the diaphragm of the mask assembly of FIG. 14;

FIG. 25 is a front elevation view of a receptacle of the mask assembly of FIG. 14;

FIG. 26 is a rear elevation view of mask assembly of FIG. 14;

FIG. 27 is an exploded front elevation view of a mask assembly according to another embodiment;

FIG. 28 is an exploded front elevation view of the mask assembly of FIG. 27;

FIG. 29 is an exploded front elevation view of the mask assembly of FIG. 27;

FIG. 30 is an exploded front elevation view of the mask assembly of FIG. 27;

FIG. 31 is a front perspective view of a seal of the mask assembly of FIG. 27;

FIG. 32 is a front elevation view of straps of the mask assembly of FIG. 27;

FIG. 33 is a front perspective view of the mask assembly of FIG. 27; and

FIG. 34 is a rear perspective view of the mask assembly of FIG. 27.

### DETAILED DESCRIPTION

As required, detailed embodiments of the present invention are disclosed herein;

however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

CPAP masks vary and include full-face masks and nasal masks. The two masks unfortunately are not always reliable, resulting in users to discontinue therapy. Sometimes a patient will discontinue therapy due to dry mouth which can lead to arousals. One cause of dry mouth is mask leakage. When air pressure is used, the air pressure will escape from any leak in the mask. One issue when using a nasal mask is that the air will go through the nose and protrude through the airway from the nose to the mouth and exit through the mouth. Escape of air pressure from the mouth leads to dry mouth which leads to the patient awakening. These effects sometimes lead to the patient discontinuing CPAP therapy. CPAP therapy can lead to a longer life and better quality of life. Meanwhile, if sleep is not corrected, it can lead to premature death.

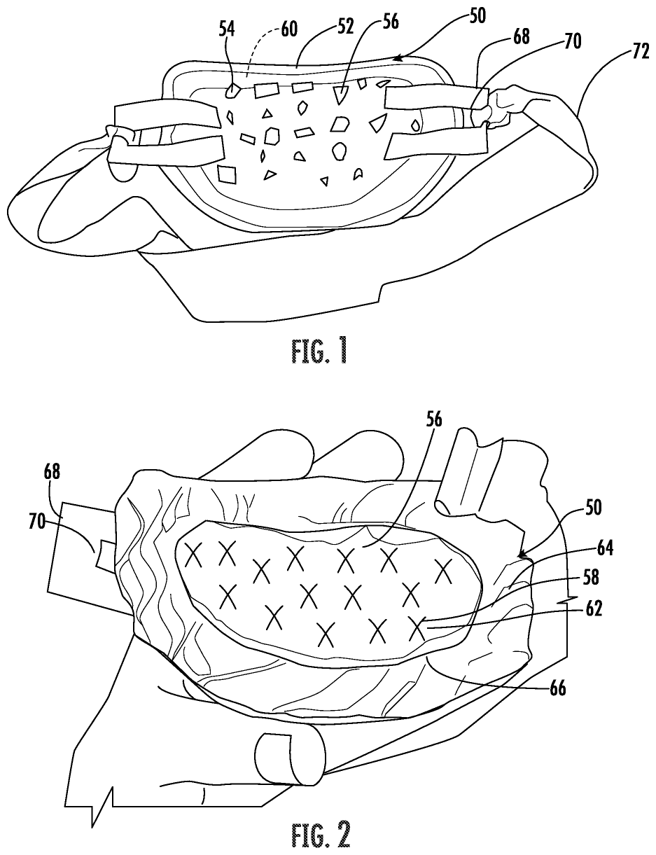

Referring to FIGS. 1 and 2, a mask assembly 50 is illustrated according to an embodiment. The mask assembly 50 is to cover a mouth of a user for use in conjunction with a nasal mask that is connected to a CPAP machine. The mask assembly 50 is an air back flow mask assembly 50. The mask assembly 50 avoids dry mouth and mouth leaks, and consequently helps avoid symptoms from dry mouth such as tooth decay, bad breath, mouth sores, infection, and the like.

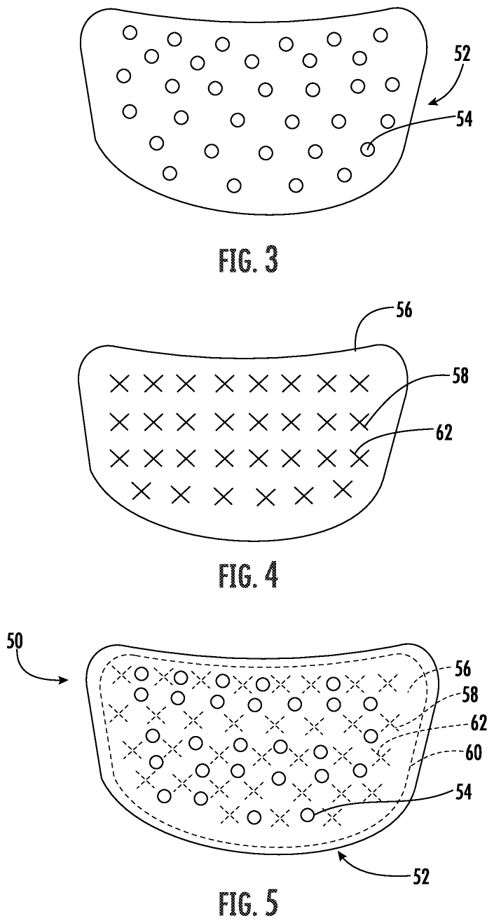

Referring now to FIGS. 1 and 3, the mask assembly 50 includes a substrate 52. The substrate 52 is sized to cover the mouth of the user. Since the mask assembly 50 is sized to be used in conjunction with a CPAP nasal mask, the substrate 52 is not sized to cover a nose of the user. The substrate 52 is formed from a rigid or semi-rigid material, such as a polymeric material, to provide adequate structure to the mask assembly 50, while permitting slight flexibility. The substrate 52 may also have a rounded shape that is symmetrical about a center bisection to accommodate the near symmetry of a common human face. The curvature may include a slight concavity at an upper region to align above the mouth of the user, while providing clearance for the nasal mask. Likewise, the curvature of the substrate 52 may include a lower convex region for extending below the mouth of the user. Additionally, the substrate 52 may be curved about the central bisection to match the curvature of a common human face curvature, from cheek to cheek.

The substrate 52 includes a plurality of ports 54 formed through the substrate 52. The ports 54 may be formed by apertures 54 that are formed through the substrate 52. The apertures 54 may be formed of any suitable size and/or pattern. The apertures 54 are positioned within an inner periphery of the substrate 52. The apertures 54 are sized to permit ingress of a fluid, particularly air, into the substrate 52, and into the mask assembly 50. The substrate 52 may be molded, stamped, cut, machined, formed by material deposition, or otherwise manufactured.

The mask assembly 50 includes a diaphragm 56 that is installed along an inside of the substrate 52 as illustrated in FIGS. 1, 2, and 4. The diaphragm 56 is formed from a thin elastomeric or rubber material and is sized to extend along the substrate 52, and over the apertures 54. The diaphragm 56 provides a valve seal over the apertures 54 to prevent egress of air from the mask assembly 50. The diaphragm 56 may formed from a sheet of material, such as latex rubber, that is cut or stamped to size. Alternatively, the diaphragm 56 may molded, extruded, or the like.

The diaphragm 56 includes a plurality of apertures 58 formed therethrough. The apertures 58 are formed as slits 58 through the diaphragm 56. The slits 58 are formed in a pattern where each slit 58 intersects with another slit 58. The slits 58 are arranged in pairs with bisecting and perpendicular intersections so that each pair of slits 58 forms a cross or X-shape. Of course, other quantities of slits 58 may form as various star shapes. The slits 58 may not bisect, and may intersect at ends of the slits to form a partial triangle or a V-shape. Any quantity and arrangement of slits 58 is contemplated.

In the depicted embodiment, each pair of intersecting slits 58 each forms a valve within the diaphragm 56. Referring to FIG. 5, the pairs of intersecting slits 58 are arranged within a perimeter of the diaphragm 56, and within the periphery of the substrate 52. The pairs of intersecting slits 58 may also be formed in a pattern such that the intersecting slits 58 do not overlap the ports 54, or are otherwise not aligned with the ports 54. The diaphragm 56 is bonded directly to the substrate 52 by application of an adhesive 60 between the diaphragm 56 and the substrate 52 around the periphery of the diaphragm 56. One suitable adhesive 60 is a silicone adhesive. The diaphragm 56 may be bonded to the substrate 52 by welding, fasteners, retainers, or any suitable retainer or fastener.

Referring now to FIGS. 1-4 and 5, the plurality of slits 58 provide a plurality of flapper valves. For example, in the depicted embodiments, each pair of slits 58 provides a flapper valve with four flaps 62. The diaphragm 56 is supported behind the substrate 52 such that the flaps 62 are prevented from flexing in a direction toward the substrate 52. This support keeps the flaps 62 closed in an exhale condition, thereby maintaining the flaps 62 closed, and consequently maintaining the slits 58 closed to prevent egress of air from the mask assembly 50. The diaphragm 56 is elastically deformable, whereby inhalation from the user provides a negative air pressure within the mask assembly 50 sufficient to deform the flaps 62 inward and away from the substrate 52. The pivoted flaps 62 open the slits 58 inward and away from the substrate 52 thereby permitting ingress of air through the ports 54 and then through the slits 58. At the end of the inhale, the material characteristics and elasticity of the diaphragm 56 permit retraction of the flaps 62 to close the slits 58. The next exhale continues to close the flaps 62, if not already closed. The next exhale is also contained within the mask assembly 50 due to the closed flaps 62. Therefore, the diaphragm 56 operates as a one-way valve or a check valve for air intake, while preventing exhaust of the exhale.

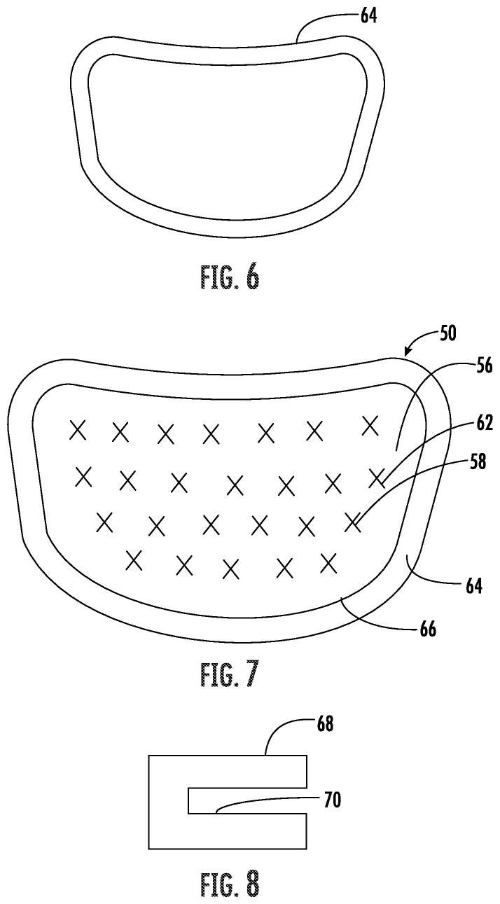

FIGS. 2, 6, and 7 illustrate a peripheral seal 64 for the mask assembly 50. The seal 64 is formed from a foam material, such as a gel foam, which is shaped to extend around an outer periphery of the diaphragm 56 and the substrate 52. The seal 64 may be cut, molded, or otherwise suitably manufactured. The seal 64 has a thickness, for example one half inch, to space the diaphragm 56 and substrate 52 away from the face of the user. The seal 64 seals the diaphragm 56 to the face of the user thereby creating a sealed air chamber 66 that is bounded by the face of the user, the seal 64, and the diaphragm 56 with the mouth of the user enclosed within the chamber 66, and the valves 58, 62 enclosed within the chamber 66. The seal 64 is bonded to the diaphragm 56 and the substrate 52 by an adhesive, such as silicone adhesive, or any suitable air impermeable bonding.

FIGS. 1 and 8-10 illustrate a pair of brackets 68. The brackets 68 are formed from a semi-rigid material, such as plastic, and are attached to the substrate 52 at opposed lateral sides of the substrate 52. The brackets 68 may be formed from a similar material as the substrate 52. The brackets 68 may be formed integral with the substrate 52. The brackets 68 may also be glued, welded, or otherwise fastened to the substrate 52. The brackets 68 cooperate with the substrate 52 to provide a pair of apertures 70 on opposed distal ends of the substrate 52.

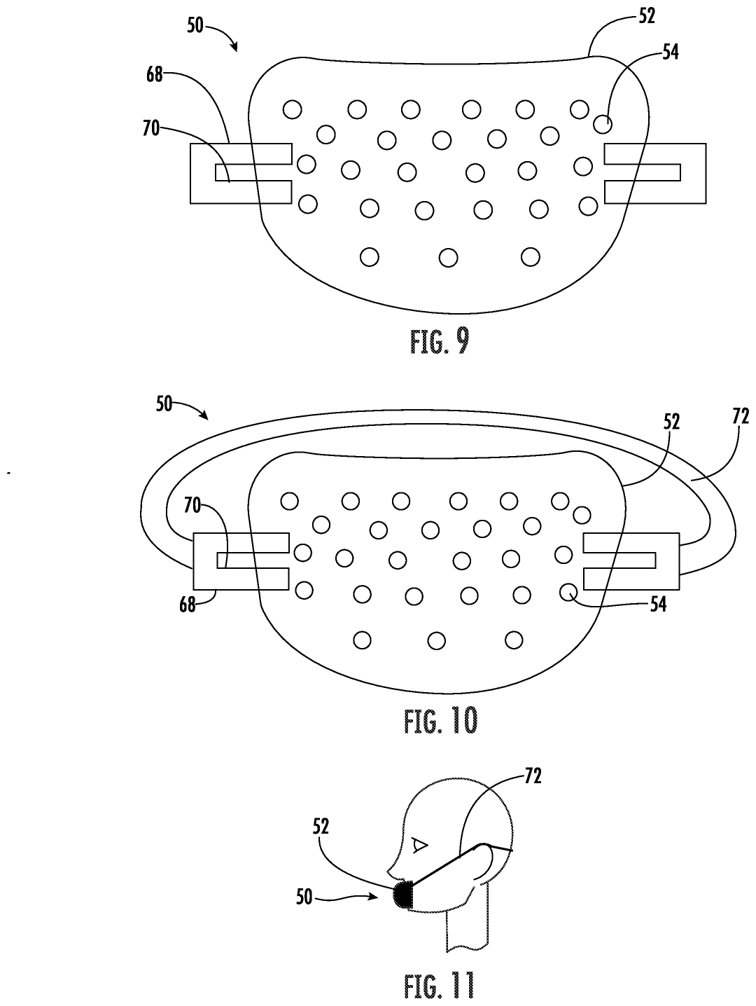

Now with reference to FIGS. 1 and 10, an elastic strap 72 is attached to the apertures 70 in the brackets 68. The strap 72 may be formed from an elastomeric or rubber material. FIG. 11 illustrates the user wearing the mask assembly 50. The substrate 52 is placed over the mouth of the user, beneath the nose, and with the seal 64 facing inward. The seal 64 contacts the face of the user and seals the air chamber 66 between the diaphragm 56, the seal 64 and the face. The strap 72 is extended around the head of the user, which may be over the ears of the user. The strap 72 maintains the mask assembly 50 upon the face of the user.

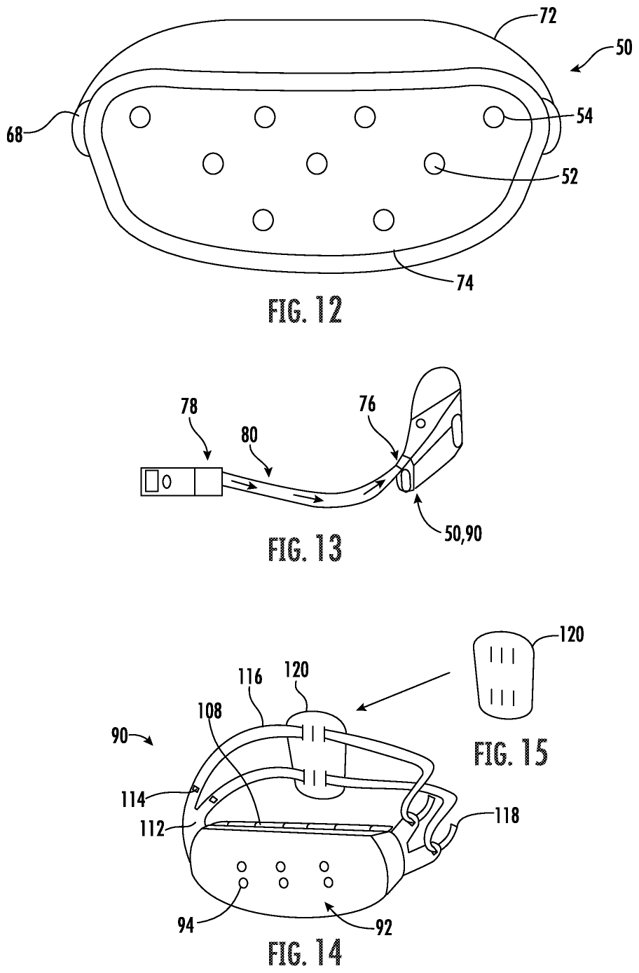

FIG. 12 illustrates the mask assembly 50 according to an embodiment. A flexible member, such as a plastically deformable wire 74, is installed on the substrate 52. The wire 74 extends offset from the periphery of the substrate 52. The wire 74 is adhered upon, welded to, or molded into, the substrate 52. The wire 74 permits the user to bend the substrate 52 and the wire 74 to match a shape and a contour of the face of the user. Alternatively, the wire 74 may be embedded within the seal 64.

The mask assembly 50 includes vents 54 and valves 58, 62 to allow air to be inhaled through but not exhaled through the mask assembly 50. This assembly 50 causes exhaled air to be directed through the nose of the user. This design of the mask assembly 50 does not pressurized of force air itself, but retains pressurized air for nasal exhalation and prevents air from being leaked from away from the mouth of the user. The prior art has provided a chin strap to attempt to close the mouth of the user. However, such chin straps have been ineffective because air was still escaping the mouth with the strapped chin. The mask assembly 50 prevents air leakage from the mouth, while maintaining the continuous pressure in the airway, and providing non-restrictive valves to permit inhalation of fresh air. The mask assembly 50 permits natural breathing patterns, whereby a user may hear its own inhalation through the mask assembly 50. By maintaining a continuous positive airway pressure, mouth leaks and dry mouth are prevented, thereby minimizing tooth plaque, tooth decay, gum disease, nighttime arousals. Additionally, improved sleep helps patients live longer and alleviates some health issues caused by sleep apnea, which may otherwise lead to high blood pressure, heart disease, stroke, brain damage, depression, and obesity.

FIG. 13 illustrates a user with a CPAP nasal mask assembly 76, as is known in the art. The nasal mask assembly 76 is sized to be worn over the nose of the user. The nasal mask assembly 76 is connected to a CPAP machine 78 by a hose 80. The CPAP machine 78 provides pressurized air, through the hose 80, and into the nasal mask assembly 76 to pressurize the airway. The nasal mask assembly 76 is utilized in conjunction with the mask assembly 50 according to the prior embodiment, or subsequent embodiments.

Referring to FIGS. 14, and 16-19, a mask assembly 90 is illustrated according to an embodiment. The mask assembly 90 is sized to cover the mouth of the user for use in conjunction with the nasal mask assembly 76 that is connected to the CPAP machine 78. The mask assembly 90 is an air back flow mask assembly 90. The mask assembly 90 avoids dry mouth and mouth leaks, and consequently helps avoid symptoms from dry mouth such as tooth decay, bad breath, mouth sores, infection, and the like.

The mask assembly 90 includes a substrate 92, which provides a housing for the mask assembly 90. The substrate 92 is sized to cover the mouth of the user. Since the mask assembly 90 is sized to be used in conjunction with a CPAP nasal mask, the substrate 92 is not sized to cover a nose of the user. The substrate 92 may be narrower in an upper center region for clearance with the nasal mask assembly 76. The substrate 92 is formed from a rigid or semi-rigid material, such as a polymeric material, to provide adequate structure to the mask assembly 90, while permitting slight flexibility. The substrate 92 may also have a rounded shape that is symmetrical about a center bisection to accommodate the near symmetry of a common human face. The curvature may include a slight concavity at an upper region to align above the mouth of the user, while providing clearance for the nasal mask. Likewise, the curvature of the substrate 92 may include a lower convex region for extending below the mouth of the user. Additionally, the substrate 92 may be curved about the central bisection to match the curvature of a common human face curvature, from cheek to cheek.

The substrate 92 includes a plurality of ports 94 formed through the substrate 92. The ports 94 may be formed by apertures 94 that are formed through the substrate 92. The apertures 94 may be formed of any suitable size and/or pattern. The apertures 94 are positioned within an inner periphery of the substrate 92. The apertures 94 are sized to permit ingress or intake of air, into the substrate 92, and into the mask assembly 90. The substrate 92 may be molded, stamped, cut, machined, formed by material deposition such as three-dimensional printing, or otherwise manufactured.

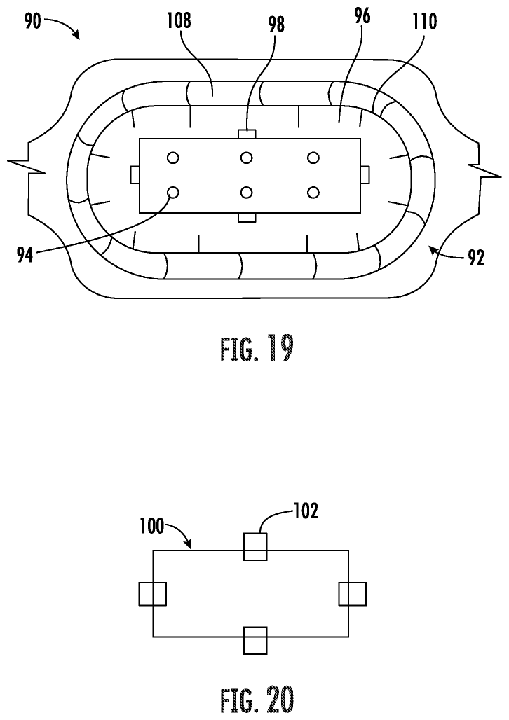

Referring now to FIGS. 19, 21, 25, and 26, a receptacle 96 is formed into an interior side of the substrate 92. The receptacle 96 overlaps the plurality of ports 94. A series of first retainers 98 (FIGS. 19 and 26) are formed around the receptacle 96. The first retainers 98 may each include an abutment edge as is known in the art. Alternatively, any suitable retainer or fastener may be utilized in the plurality of first retainers 98.

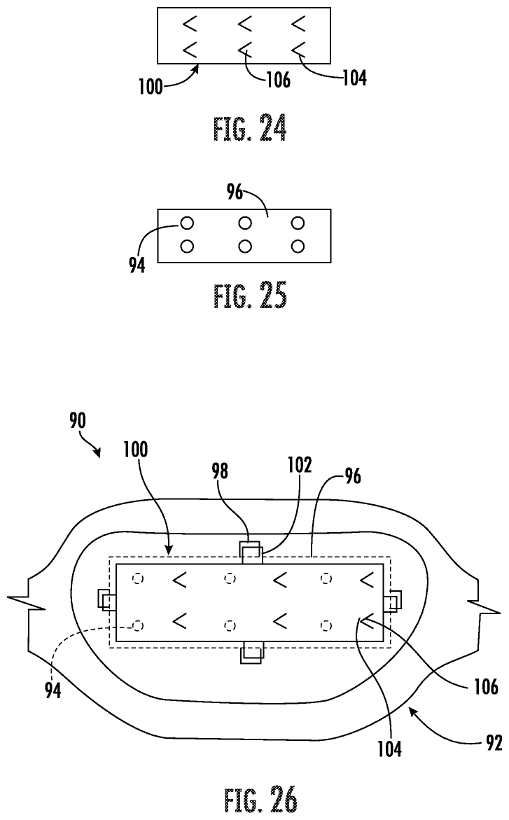

The mask assembly 90 includes a diaphragm 100 illustrated in FIGS. 20 and 24, that is installed in the receptacle 96 of the substrate 92 as illustrated in FIG. 26. The diaphragm 100 is formed from a thin elastomeric or rubber material and is sized to extend along the substrate 92, and over the apertures 94. The diaphragm 100 provides a valve seal over the apertures 94 to prevent egress or exhaust of air from the mask assembly 90. The diaphragm 100 may formed from a sheet of material, such as latex rubber, that is cut or stamped to size. Alternatively, the diaphragm 100 may molded, extruded, or the like.

The diaphragm includes a series of second retainers 102 as illustrated in FIGS. 20 and 26. The second retainers 102 are sized to be received in the first retainers 98 to fasten and retain the diaphragm 100 into the receptacle 96. For example, the second retainers 102 may project from the diaphragm 100 with a leading edge and an abutment edge, as is known in the art. Upon installation of the diaphragm 100 into the receptacle 96, the leading edges of the second retainers 102 engage the abutment edges of the first retainers 98 to flex until the abutment edges of the second retainers 102 align and expand into contact with the abutment edges of the first retainers 98, as is known in the art, to retain the diaphragm 100 into the receptacle 96. Such retainer arrangement 98, 102 is often referred to as snap-fit fasteners, or the like. Of course any suitable retainer or fastener arrangement may be utilized. The retainer arrangement 98, 102 permits removal and replacement of the diaphragm 100 into the receptacle 96.

As illustrated in FIGS. 24 and 26, the diaphragm 100 includes a plurality of apertures 104 formed therethrough. The apertures 104 are formed as slits 104 through the diaphragm 100. The slits 104 are formed in a pattern where each slit 104 intersects with another slit 104. The slits 104 are arranged in pairs with angled intersections at ends of the slits 104 so that each pair of slits 104 forms a partial triangle or a V-shape. Any quantity and arrangement of slits 104 is contemplated. In the depicted embodiment, each pair of intersecting slits 104 each forms a valve within the diaphragm 100. The pairs of intersecting slits 104 are arranged within a perimeter of the diaphragm 100. The pairs of intersecting slits 104 may also be formed in a pattern such that the intersecting slits 104 do not overlap the ports 94, or are otherwise not aligned with the ports 94.

The plurality of slits 104 provide a plurality of flapper valves. For example, in the depicted embodiments, each pair of slits 104 provides a flapper valve with one flap 106. The diaphragm 100 is supported behind the substrate 92 such that the flaps 106 are prevented from flexing in a direction toward the substrate 92. This support keeps the flaps 106 closed in an exhale condition, thereby maintaining the flaps 106 closed, and consequently maintaining the slits 104 closed to prevent egress of air from the mask assembly 90. The diaphragm 100 is elastically deformable, whereby inhalation from the user provides a negative air pressure within the mask assembly 90 sufficient to deform the flaps 106 inward and away from the substrate 92. The pivoted flaps 106 open the slits 104 inward and away from the substrate 92 thereby permitting ingress of air through the ports 94 and then through the slits 104. At the end of the inhale, the material characteristics and elasticity of the diaphragm 100 permit retraction of the flaps 106 to close the slits 104. The next exhale continues to close the flaps 106, if not already closed. The next exhale is also contained within the mask assembly 90 due to the closed flaps 106. Therefore, the diaphragm 100 operates as a one-way valve or a check valve for air intake, while preventing exhaust of the exhale. According to an embodiment, the receptacle 96 is flat, and the diaphragm 100 is flat for a sealed fluid cooperation.

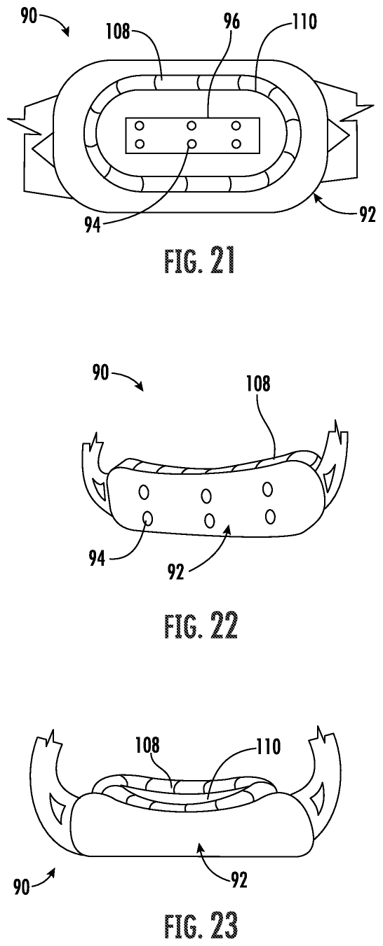

FIGS. 14, 16, 18, 19, 21, 22, and 23 illustrate a peripheral seal 108 for the mask assembly 90. The seal 108 is formed from a foam material, such as a gel foam, which is shaped to extend around an outer periphery of the diaphragm 100 and the substrate 92. The seal 108 may be cut, molded, or otherwise suitably manufactured. The seal 108 has a thickness, for example one half inch, to space the diaphragm 100 and substrate 92 away from the face of the user. The seal 108 seals the diaphragm 100 to the face of the user thereby creating a sealed air chamber 110 (FIGS. 19, 21, and 23) that is bounded by the face of the user, the seal 108, the diaphragm 100, and the substrate 92, with the mouth of the user enclosed within the chamber 110, and the valves 104, 106 enclosed within the chamber 110. The seal 108 is bonded to the diaphragm 100 by an adhesive, such as silicone adhesive, or any suitable air impermeable bonding. Raised lateral sides and a bottom of the substrate 92 provide a contour to mate the seal 108 to the user while expanding the chamber 110.

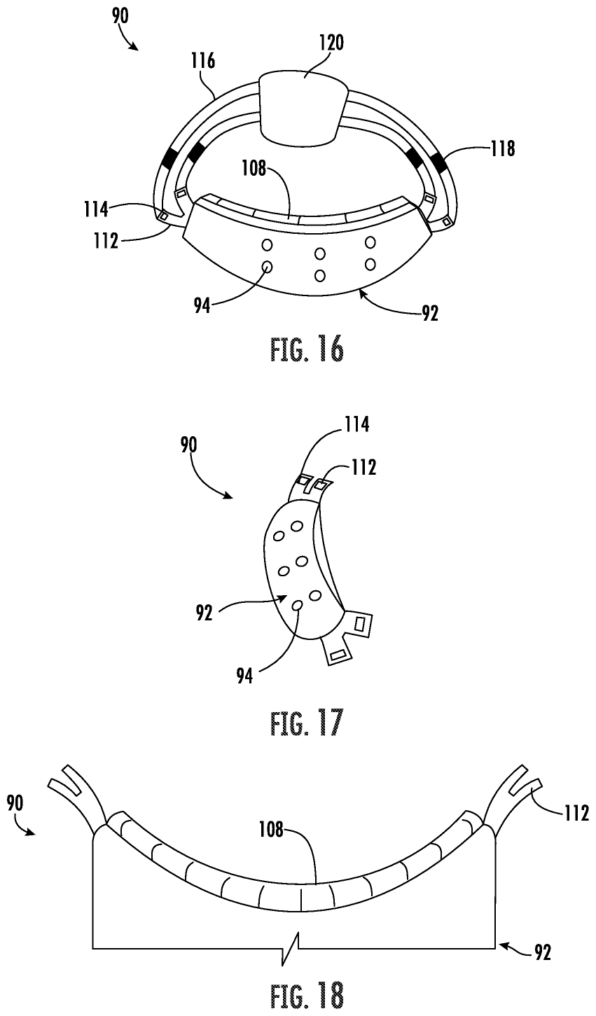

FIGS. 14, 16, 17, and 18 illustrate pairs of brackets 112. The brackets 112 are formed integral with the substrate 92. The brackets 112 cooperate with the substrate 92 to provide pairs of apertures 114 on opposed distal ends of the substrate 92. Now with reference to FIGS. 14 and 16, elastic straps 116 are attached to the apertures 114 in the brackets 112. The straps 116 may be formed from an elastomeric or rubber material. Fasteners 118 are installed on the straps 116 to permit adjustment of the straps 116. The fasteners 118 may be buckles, hook and loop fasteners, or the like. An adjustable headrest 120 is illustrated in FIGS. 14-16. The headrest 120 can be utilized in cooperation with the straps 116 to distribute a load from the tensioned straps 116 to an expanded surface of the head of the user for comfort and support.

FIG. 13 illustrates the user wearing the mask assembly 90. The substrate 92 is placed over the mouth of the user, beneath the nose, and with the seal 108 facing inward. The seal 108 contacts the face of the user and seals the air chamber 110 between the diaphragm 100, the seal 108, the substrate 92 and the face. The straps 116 are extended around the head of the user, which may be over the ears of the user. The straps 116 maintain the mask assembly 90 upon the face of the user.

The mask assembly 90 includes vents 94 and valves 104, 106 to allow air to be inhaled through but not exhaled through the mask assembly 90. This assembly 90 causes exhaled air to be directed through the nose of the user. This design of the mask assembly 90 does not pressurized of force air itself, but retains pressurized air for nasal exhalation and prevents air from being leaked from away from the mouth of the user. The mask assembly 90 prevents air leakage from the mouth, while maintaining the continuous pressure in the airway, and providing non-restrictive valves to permit inhalation of fresh air. The mask assembly 90 permits natural breathing patterns, whereby a user may hear its own inhalation through the mask assembly 90. By maintaining a continuous positive airway pressure, mouth leaks and dry mouth are prevented, thereby minimizing tooth plaque, tooth decay, gum disease, nighttime arousals. Additionally, improved sleep helps patients live longer and alleviates some health issues caused by sleep apnea, which may otherwise lead to high blood pressure, heart disease, stroke, brain damage, depression, and obesity.

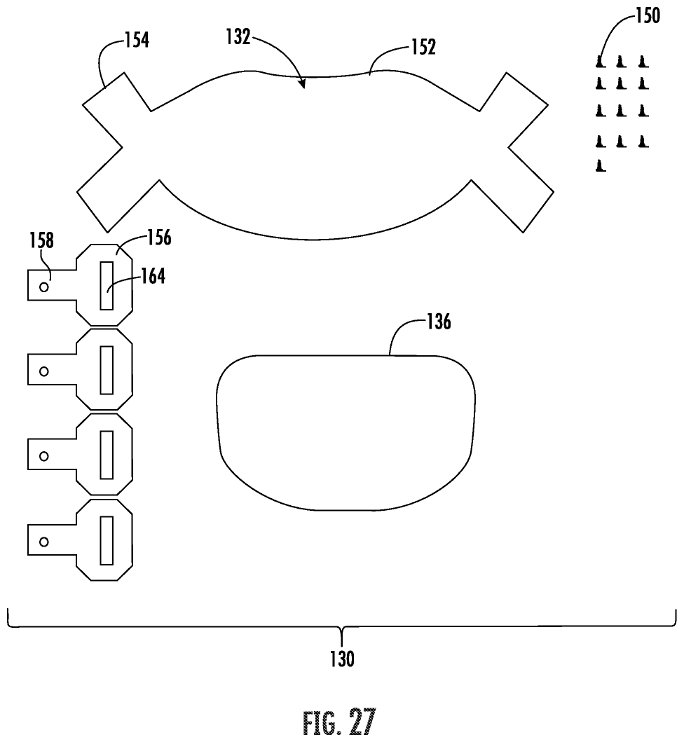

Referring to FIGS. 27, 28, 33, and 34, a mask assembly 130 is illustrated according to another embodiment. The mask assembly 130 is sized to cover the mouth of the user for use in conjunction with the nasal mask assembly 76 that is connected to the CPAP machine 78. The mask assembly 130 is an air back flow mask assembly 130. The mask assembly 130 avoids dry mouth and mouth leaks, and consequently helps avoid symptoms from dry mouth such as tooth decay, bad breath, mouth sores, infection, and the like.

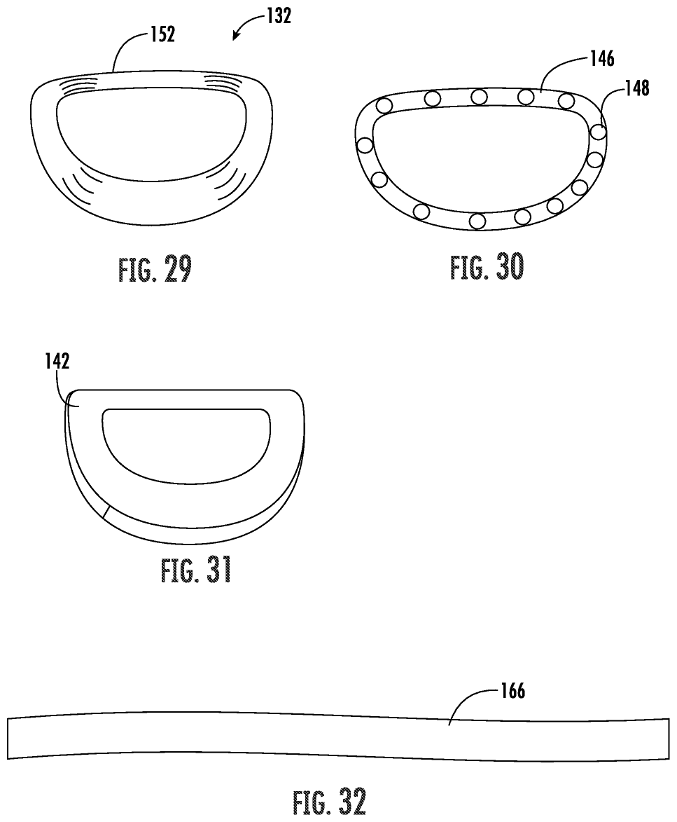

Referring now to FIGS. 27, 28, 33, and 34, the mask assembly 130 includes a substrate 132. The curvature of a central region 152 of the substrate is illustrated in FIG. 29. The substrate 132 is sized to cover the mouth of the user. Since the mask assembly 130 is sized to be used in conjunction with the CPAP nasal mask assembly 76, the substrate 132 is not sized to cover the nose of the user. The substrate 132 is formed from a rigid or semi-rigid material, such as a polymeric material, to provide adequate structure to the mask assembly 130, while permitting slight flexibility. The substrate 132 may also have a rounded shape that is symmetrical about a center bisection to accommodate the near symmetry of a common human face. The curvature may include a slight concavity at an upper region to align above the mouth of the user, while providing clearance for the nasal mask assembly 76. Likewise, the curvature of the substrate 132 may include a lower convex region for extending below the mouth of the user. Additionally, the substrate 132 may be curved about the central bisection to match the curvature of a common human face curvature, from cheek to cheek.

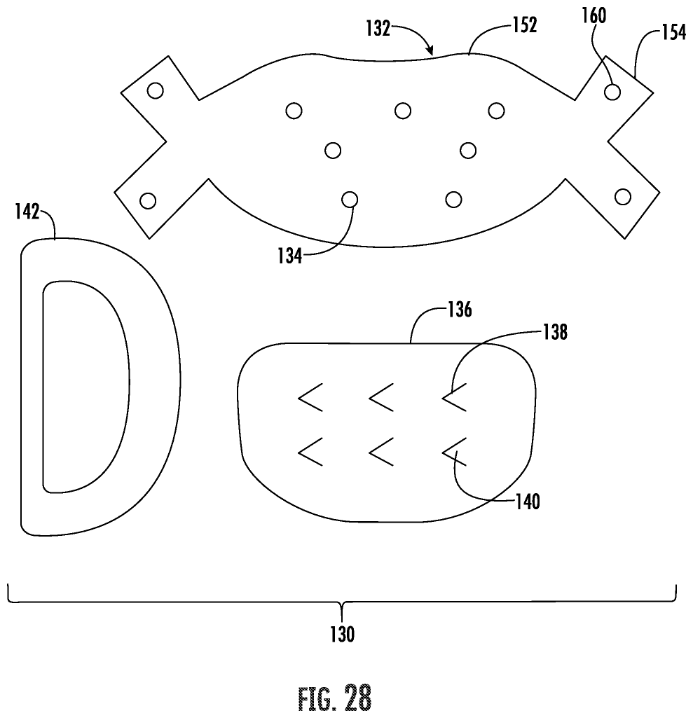

As illustrated in FIGS. 28 and 33, the substrate 132 includes a plurality of ports 134 formed through the substrate 132. The ports 134 may be formed by apertures 134 that are formed through the substrate 132. The apertures 134 may be formed of any suitable size and/or pattern. The apertures 134 are positioned within an inner periphery of the substrate 132. The apertures 134 are sized to permit ingress of a fluid, particularly air, into the substrate 132, and into the mask assembly 130. The substrate 132 may be molded, stamped, cut, machined, formed by material deposition, or otherwise manufactured. The apertures 134 may be drilled, stamped, molded or the like.

The mask assembly 130 includes a diaphragm 136 that is installed along an inside of the substrate 132 as illustrated in FIGS. 27, 28 and 34. The diaphragm 136 is formed from a thin elastomeric or rubber material and is sized to extend along the substrate 132, and over the apertures 134. The diaphragm 136 provides a valve seal over the apertures 134 to prevent egress of air from the mask assembly 130. The diaphragm 136 may formed from a sheet of material, such as latex rubber, that is cut or stamped to size. Alternatively, the diaphragm 136 may molded, extruded, or the like.

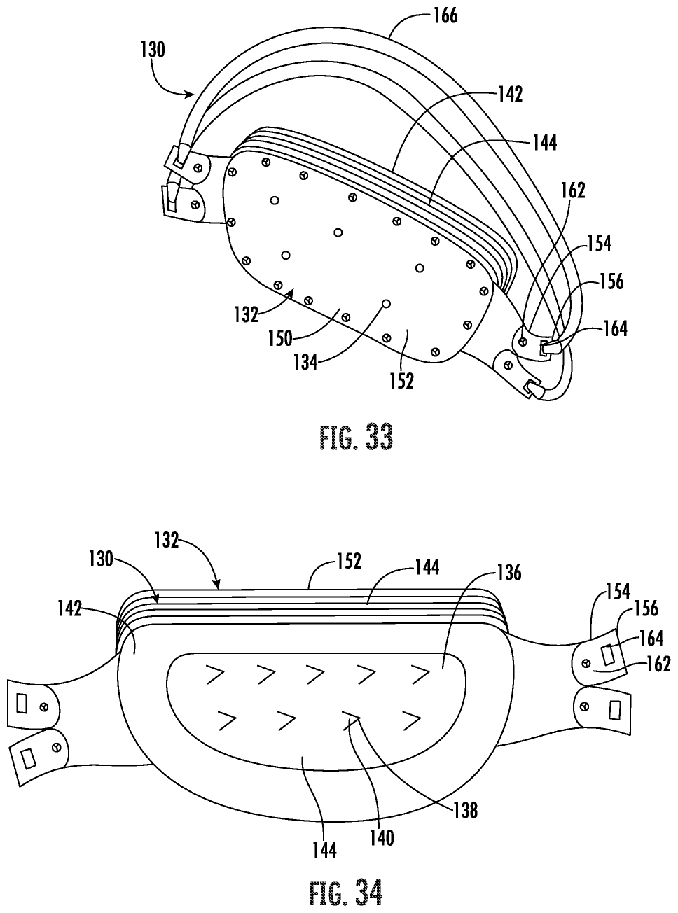

The diaphragm 136 includes a plurality of apertures 138 formed therethrough as illustrated in FIGS. 28 and 34. The apertures 138 are formed as slits 138 through the diaphragm 136. The slits 138 are formed in a pattern where each slit 138 intersects with another slit 138. The slits 138 are arranged in pairs with angled intersections so that each pair of slits 138 forms a partial triangle or a V-shape. Any quantity and arrangement of slits 138 is contemplated.

In the depicted embodiment, each pair of intersecting slits 138 each forms a valve within the diaphragm 136. The pairs of intersecting slits 138 are arranged within a perimeter of the diaphragm 136, and within the periphery of the substrate 132. The pairs of intersecting slits 138 may also be formed in a pattern such that the intersecting slits 138 do not overlap the ports 134, or are otherwise not aligned with the ports 134. The diaphragm 136 is bonded directly to the substrate 132 by application of an adhesive between the diaphragm 136 and the substrate 132 around the periphery of the diaphragm 136. One suitable adhesive is a silicone adhesive. The diaphragm 136 may be bonded to the substrate 132 by welding, fasteners, retainers, or any suitable retainer or fastener.

The plurality of slits 138 provide a plurality of flapper valves. For example, in the depicted embodiments, each pair of slits 138 provides a flapper valve with one flap 140. The diaphragm 136 is supported behind the substrate 132 such that the flaps 140 are prevented from flexing in a direction toward the substrate 132. This support keeps the flaps 140 closed in an exhale condition, thereby maintaining the flaps 140 closed, and consequently maintaining the slits 138 closed to prevent egress of air from the mask assembly 130. The diaphragm 136 is elastically deformable, whereby inhalation from the user provides a negative air pressure within the mask assembly 130 sufficient to deform the flaps 140 inward and away from the substrate 132. The pivoted flaps 140 open the slits 138 inward and away from the substrate 132 thereby permitting ingress of air through the ports 134 and then through the slits 138. At the end of the inhale, the material characteristics and elasticity of the diaphragm 136 permit retraction of the flaps 140 to close the slits 138. The next exhale continues to close the flaps 140, if not already closed. The next exhale is also contained within the mask assembly 130 due to the closed flaps 140. Therefore, the diaphragm 136 operates as a one-way valve or a check valve for air intake, while preventing exhaust of the exhale.

FIGS. 28, 31, 33, and 34 illustrate a peripheral seal 142 for the mask assembly 130. The seal 142 is formed from a foam material, such as a gel foam, which is shaped to extend around an outer periphery of the diaphragm 136 and the substrate 132. The seal 142 may be cut, molded, or otherwise suitably manufactured.

FIGS. 30, 33, and 34 illustrate a plurality of rubber gaskets 146. As depicted in FIG. 30, a plurality of apertures 148 are formed through the rubber gaskets 146. The gaskets 146 may be formed from sheets of an elastomeric material, or may be molded. Referring again to FIGS. 27 and 33, a plurality of screws 150 are installed into an exterior of the substrate 132, along a periphery of the substrate 132 to fasten the rubber gaskets 146 to the substrate 132.

With reference again to FIGS. 33 and 34, the seal 142 has a thickness for flexibility, and to space the diaphragm 136 and substrate 132 away from the face of the user. The seal 142 seals the rubber gaskets 146 and the diaphragm 136 to the face of the user thereby creating a sealed air chamber 144 (FIG. 34) that is bounded by the face of the user, the seal 142, the rubber gaskets 146, and the diaphragm 136 with the mouth of the user enclosed within the chamber 144, and the valves 138, 140 enclosed within the chamber 144. The seal 142 is bonded to the distal rubber gasket 146 by an adhesive, such as silicone adhesive, or any suitable air impermeable bonding.

FIGS. 27, 28, 33, and 34 illustrate two pairs of tabs 154 extending laterally outboard from the central region 152 of the substrate 132. FIGS. 27, 33, and 34 illustrate two pairs of brackets 156. The brackets 156 are formed from a semi-rigid material, such as plastic. As illustrated in FIG. 27, the brackets 156 include a plurality of fastener apertures 158 formed therethrough to align with tab apertures 160 in the tabs 154 of the substrate 132, which are illustrated in FIG. 28. FIGS. 33 and 34 illustrate that a plurality of fasteners 162, such as screws or rivets, are installed in the apertures 158 of the brackets 156 and the tab apertures 160 of the tabs 154 of the substrate 132 to fasten the brackets 156 to the tabs 154 of the substrate 132. The brackets 156 may be formed from a similar material as the substrate 132. The brackets 156 may be formed integral with the substrate 132. The brackets 156 may also be glued, welded, or otherwise fastened to the substrate 132. The brackets 156 cooperate with the substrate 132, and each provide an aperture 164 on opposed distal ends of the substrate 132.

Now with reference to FIGS. 32 and 33, a pair of elastic straps 166 are attached to the apertures 164 in the brackets 156. The straps 166 may be formed from an elastomeric or rubber material. The substrate 132 is placed over the mouth of the user, beneath the nose, and with the seal 142 facing inward. The seal 142 contacts the face of the user and seals the air chamber 144 between the diaphragm 136, the seal 142 and the face. The straps 166 are extended around the head of the user, which may be over the ears of the user. The straps 166 maintain the mask assembly 130 upon the face of the user.

The mask assembly 130 includes vents 134 and valves 138, 140 to allow air to be inhaled through but not exhaled through the mask assembly 130. This assembly 130 causes exhaled air to be directed through the nose of the user. This design of the mask assembly 130 does not pressurized of force air itself, but retains pressurized air for nasal exhalation and prevents air from being leaked from away from the mouth of the user. The prior art has provided a chin strap to attempt to close the mouth of the user. However, such chin straps have been ineffective because air was still escaping the mouth with the strapped chin. The mask assembly 130 prevents air leakage from the mouth, while maintaining the continuous pressure in the airway, and providing non-restrictive valves to permit inhalation of fresh air. The mask assembly 130 permits natural breathing patterns, whereby a user may hear its own inhalation through the mask assembly 130. By maintaining a continuous positive airway pressure, mouth leaks and dry mouth are prevented, thereby minimizing tooth plaque, tooth decay, gum disease, nighttime arousals. Additionally, improved sleep helps patients live longer and alleviates some health issues caused by sleep apnea, which may otherwise lead to high blood pressure, heart disease, stroke, brain damage, depression, and obesity.

While various embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A mask assembly comprising:
a substrate sized to cover a mouth of a user, wherein a plurality of ports is formed through the substrate;
a valve seal mounted to the substrate, the valve seal having a plurality of valves formed through the valve seal; and
a peripheral seal mounted to the substrate to seal around the valve to seal the substrate and the valve seal to a face of the user around the mouth of the user.

2. The mask assembly of claim 1, wherein the ports in the substrate are formed as plurality of apertures.

3. The mask assembly of claim 1, wherein the valves are formed through the valve seal as a plurality of apertures.

4. The mask assembly of claim 3, wherein the plurality of apertures in the valve seal are not aligned with the plurality of ports in the substrate.

5. The mask assembly of claim 3 wherein the plurality of apertures is formed through the valve seal as a plurality of slits; and
wherein each slit of the plurality of slits intersects another slit of the plurality of slits.

6. The mask assembly of claim 5, wherein the intersecting slits provide a series of flaps; and
wherein the valve seal is formed from a flexible material to permit elastic deformation of the flaps to open the valves, and retraction of the flaps to close the valves.

7. The mask assembly of claim 6, wherein the valve seal is displaced along the substrate such that the substrate prevents deformation of the flaps in an egress direction of fluid flow, while permitting elastic deformation of the flaps in an ingress direction of fluid flow.

8. The mask assembly of claim 1, further comprising a deformable member attached to the substrate to permit shaping of the substrate to the face of the user.

9. The mask assembly of claim 1, wherein the substrate is sized to not cover a nose of the user.

10. The mask assembly of claim 1, wherein the valve seal further comprises a diaphragm.

11. The mask assembly of claim 10, further comprising a retainer cooperating with the diaphragm and the substrate to retain the diaphragm to the substrate for removal and replacement of the diaphragm.

12. A mask assembly comprising:
a substrate sized to be placed over a mouth of a user, wherein a port is formed through the substrate;
a valve seal mounted to the substrate, the valve seal having a valve formed through the valve seal, the valve seal cooperating with the substrate to permit ingress of fluid through the port and the valve seal, while preventing egress of fluid through the valve seal and the port; and
a peripheral seal mounted to the substrate to seal around the valve to seal the substrate and the valve seal to a face of the user around the mouth of the user.

13. The mask assembly of claim 12, wherein the port further comprises a plurality of ports.

14. The mask assembly of claim 12, wherein the valve further comprises a plurality of ports.

15. The mask assembly of claim 12, wherein the valve is formed through the valve seal as a plurality of intersecting slits;
wherein the intersecting slits provide a series of flaps; and
wherein the valve seal is formed from a flexible material to permit elastic deformation of the flaps to open the valve, and retraction of the flaps to close the valve.

16. The mask assembly of claim 15, wherein the valve seal is displaced along the substrate such that the substrate prevents deformation of the flaps in an egress direction of fluid flow, while permitting elastic deformation of the flaps in an ingress direction of fluid flow.

17. The mask assembly of claim 12, wherein the valve seal further comprises a diaphragm.

18. The mask assembly of claim 17, further comprising a retainer cooperating with the diaphragm and the substrate to retain the diaphragm to the substrate for removal and replacement of the diaphragm.

19. A mask assembly comprising:
a substrate sized to be placed over a mouth of a user, wherein a port is formed through the substrate;
a peripheral seal mounted to the substrate to seal the substrate to a face of the user around the mouth of the user to provide an enclosed chamber around the mouth of the user; and
a check valve mounted to the substrate to permit fluid ingress into the chamber, while preventing fluid egress from the chamber.

20. The mask assembly of claim 19, further comprising a retainer cooperating with the check valve and the substrate to retain the check valve to the substrate for removal and replacement of the check valve.
